(19) United States
(12) Patent Application Publication
(10) Pub. No.: US 2006/0183714 A1
(43) Pub. Date: Aug. 17, 2006

(54) 3-Alpha-glycosyl alpha, alpha-trehalose compound, process for producing the same, and use

(76) Inventors: Tomoyuki Nishimoto, Okayama (JP); Hikaru Watanabe, Okayama (JP); Shigeharu Fukuda, Okayama (JP); Toshio Miytake, Okayama (JP)

Correspondence Address: BROWDY AND NEIMARK, P.L.L.C.;624 NINTH STREET, NW, SUITE 300, WASHINGTON, DC, 20001-5303, US

(21) Appl. No.: 10/565,083

(22) PCT Filed: Jul. 16, 2004
(86) PCT No.: PCT/JP04/10225

§ 371 Date: Jan. 18, 2006

(30) Foreign Application Priority Data
Jul. 18, 2003 (JP) 2003-276632

(51) Int. Cl. A61K 31/715; C12P 19/04; C08B 37/00

(52) U.S. Cl. 514/61; 536/123; 435/101

(57) ABSTRACT

An object of the present invention is to provide novel saccharides, 3-α-glycosyl α,α-trehalose having a 3-α-glucosyl α,α-trehalose structure represented by the chemical formula 1 within their molecules, their preparation and use. The present invention attains the above object by providing 3-α-glycosyl α,α-trehaloses having a 3-α-glucosyl α,α-trehalose structure represented by the chemical formula 1 within their molecules, their preparation and use.
O-α-D-Glcp-(1→3)-O-α-D-Glcp-(1→1)-α-D-Glcp Chemical formula 1

### TECHNICAL FIELD

The present invention relates to novel saccharides, 3-α-glycosyl α,α-trehaloses, their preparation and use, particularly, to 3-α-glycosyl α,α-trehaloses having a 3-α-glucosyl α,α-trehalose structure, represented by the chemical formula 1, within their molecules, their preparation and use.
O-α-D-Glcp-(1→3)-O-α-D-Glcp-(1→1)-α-D-Glcp Chemical formula 1

### BACKGROUND ART

α,α-Trehalose is a non-reducing disaccharide where two glucose molecules are bound via the α,α-1,1 glucosidic linkage. Although the amount of the saccharide is low, the saccharide extensively exists in nature, for example, in fungi, yeasts, bacteria, mushrooms, higher plants, insects, and the like. Because of its non-reducibility, α,α-trehalose causes no Maillard reaction (aminocarbonyl reaction) with substances having amino groups such as amino acids and proteins. The saccharide causes no deterioration of substance comprising amino acid, and is a stable saccharide itself. Therefore, α,α-trehalose can be used and processed without fear of browning and deteriorating and has been expected its uses in a various fields. Researches about the functions of α,α-trehalose are now being in progress. However, under these circumstances, researches about saccharide-derivatives of α,α-trehalose, as saccharides which can be expected to have novel functions not exhibited by α,α-trehalose, have been actively performed.

There are many reports on the synthesis of saccharide-derivatives of α,α-trehalose by enzymatic methods. Japanese Patent Kokai No. 304,882/98 and Chaen et al., “Journal of Applied Glycoscience”, (Japan), 1999, Vol.46, pp.423-429 disclose a oligosaccharide having a structure of binding α,α-trehalose and glucose via the α-1,2 glucosidic linkage, 2-O-α-glucosyl α,α-trehalose (aka α-kojibiosyl α-D-glucoside or selaginose), formed from α,α-trehalose by glucosyl-transferring actions of trehalose phosphorylase and kojibiose phosphorylase.

European Patent Publication No. 0606753 A2 discloses oligosaccharides having structures of binding α,α-trehalose and maltooligosaccharides such as maltose, maltotriose, or the like via the α-1,4 glucosiduc linkage, for example, 4-α-glycosyl α,α-trehaloses having a trehalose structure at the end of molecule, which have a starch-like structure constructed by binding glucoses via the α-1,4 glucosidic linkages and which are formed by allowing a non-reducing saccharide-forming enzyme to act on an aqueous solution containing starch or partial starch hydrolyzate (abbreviated as “starchy substance”, hereinafter), such as 4-α-D-glucosyl α,α-trehalose, 4-α-maltosyl α,α-trehalose, 4-α-maltotriosyl α,α-trehalose, 4-α-maltotetraosyl α,α-trehalose, and the like. Further, Kurimoto et al., “Bioscience Biotechnology Biochemistry”, (Japan), 1997, Vol.61, No.7, pp.1146-1149, discloses α-maltosyl α-D-glucoside, α-maltotriosyl α-D-glucoside, α-maltosyl α-maltoside, α-maltotriosyl α-maltoside, α-maltotriosyl α-maltotrioside, and the like, which are formed from α,α-trehalose and cyclomaltohexaose by the saccharide-transferring action of cyclodextrin glucanotransferase (CGTase) originated from Bacillus stearothermophilus.

With regard to oligosaccharides having structures of binding α,α-trehalose and glucoses via the α-1,6 glucosidic linkages, Ajisaka et al., “Carbohydrate Research”, (Netherlands), 1990, Vol.199, pp.227-234, discloses α-isomaltosyl α-D-glucoside formed from α,α-trehalose and glucose by the condensation reaction of α-glucosidase from Saccharomyces sp. or glucoamylase from Rhizopus niveus. Further, Kim et al., “Denpun Kagaku (in Japanese)”, (Japan), 1993, Vol.40, No.3, 227-234, discloses α-isomaltosriosyl α,α-trehalose formed from dextran and α,α-trehalose by the glucosyl-transferring action of isomaltodextranase from Arthrobacter glibiformis. In addition, Kurimoto et al. , “Bioscience Biotecnology Biochemistry”, (Japan), 1997, Vol.61, No.4, pp.699-703 and Japanese Patent Kokai No. 217,784/96 disclose α-isomaltosyl α-isomaltoside formed from α,α-trehalose and maltotetraose by the glucosyl-transferring action of α-glucosidase from Aspergillus niger.

However, saccharides having structures of binding α,α-trehalose and other saccharides via the α-1,3 glucosidic linkage have been unknown yet.

With regard to oligosaccharides having α-1,3 glucosidic linkage within their molecules, nigerose, a reducing disaccharide where two glucose molecules are bound via the α-1,3 glucosidic linkage, and nigerooligosaccharides, having a structure of binding glucose with glucose residue at the non-reducing ends of maltooligosaccharides via the α-1,3 glucosidic linkage, have been known (see Japanese Patent Kokai Nos. 59,559/95 and 299,095/97). As disclosed in Japanese Patent Kokai No.52,834/97, it is known that saccharides having nigerose as a structural unit have a strong immune-activating effect as well as functions as sweetener such as mild sweetness and taste-improving effect. However, since both nigerose and nigerooligosaccharide have reducibilities, they have disadvantages of easily causing the browning reaction with amino acids and causing the deterioration in the processing of foods.

Under these circumstances, the development of non-reducing oligosaccharides having a α,α-trehalose structure and a nigerose structure within a molecule, i.e., 3-α-glycosyl α,α-trehaloses having a 3-α-glucosyl α,α-trehalose structure represented by the chemical formula 1 within their molecules, for example, saccharides having a structure of binding α,α-trehalose and other saccharides via the α-1,3 glucosidic linkages such as3-α-glucosyl α,α-trehalose, 3-α-isomaltosyl α,α-trehalose, and the like, has been desired.

### DISCLOSURE OF INVENTION

An object of the present invention is to establish novel saccharides, 3-α-glycosyl α,α-trehaloses having a 3-α-glucosyl α,α-trehalose structure represented by the chemical formula 1 (abbreviated as “3-α-glycosyl α,α-trehaloses”, hereinafter), and a process for producing them, and to provide uses thereof.

The present inventors have been extensively studied on novel saccharides, 3-α-glycosyl α,α-trehaloses and their preparation to attain the above object. As a result, the present inventors have found novel saccharides, 3-α-isomaltosyl α,α-trehalose represented by the chemical formula 2 and 3-α-glucosyl α,α-trehalose represented by the chemical formula 3. The present inventors have also found that various other 3-α-glycosyl α,α-trehaloses can be easily synthesized by transferring other saccharides to these-novel saccharides. The present inventors accomplished the present invention by establishing saccharides comprising 3-α-glycosyl α,α-trehalose and processes for producing them. In addition, the present inventors accomplished the present invention by establishing compositions such as foods and beverages, cosmetics, and pharmaceuticals, comprising these saccharides or saccharide compositions comprising them.
O-α-D-Glcp-(1→6)-O-α-D-Glcp-(1→3)-O-α-D-Glcp-(1→1)-α-D-Glcp Chemical formula 2
O-α-D-Glcp-(1→3)-O-α-D-Glcp-(1→1)-α-D-Glcp Chemical formula 3

3-α-Glycosyl α,α-trehaloses of the present invention are novel saccharides that have been ever unknown. Since the saccharides have both α,α-trehalose structure and nigerose structure within their molecules, the saccharide is expected to have various functions. Further, the present invention, which provides 3-α-glycosyl α,α-trehaloses, their preparation and use, is a useful invention that greatly contributes to this art.

### BRIEF DESCRIPTION OF DRAWINGS

FIG. 1 shows a ¹H-NMR spectrum of a purified Transferred saccharide A.

FIG. 2 shows a ¹³C-NMR spectrum of a purified Transferred saccharide A.

FIG. 3 shows a structure of 3-α-isomaltosyl α,α-tehalose.

FIG. 4 shows a ¹H-NMR spectrum of a purified Partial hydrolyzate B.

FIG. 5 shows a ¹³C-NMR spectrum of a purified Partial hydrolyzate B.

FIG. 6 shows a structure of 3-α-glucosyl α,α-trehalose.

### EXPLANATION OF SYMBOLS

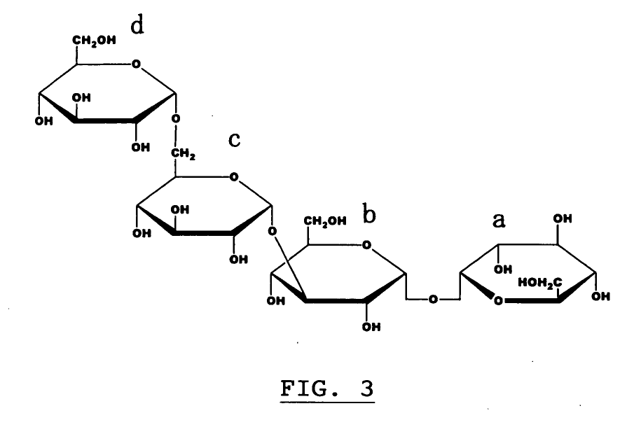

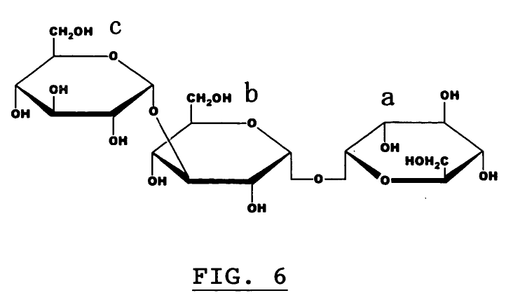

Symbols, a, b, c, and d in FIGS. 3 and 6 mean a glucosyl residue, respectively, and the symbols correspond to glucosyl residues a, b, c, and d in Tables 4 and 6.

### BEST MODE FOR CARRYING OUT THE INVENTION

“3-α-Glycosyl α,α-trehaloses” as referred to as in the present invention means all saccharides having a 3-α-glucosyl α,α-trehalose structure within its molecules. The saccharides are non-reducing saccharides having both α,α-trehalose structure and nigerose structure within their molecules.

3-α-Glycosyl α,α-trehaloses of the present invention are not restricted by their origin and a process for producing them as far as they have a 3-α-glucosyl α,α-trehalose structure represented by the aforesaid chemical formula 1 within their molecules. If they exist in nature, they can be also used in the present invention. Also, saccharides synthesized by chemical or enzymatic methods can be arbitrarily used. Both 3-α-isomaltosyl α,α-trehalose represented by the chemical formula 2 (abbreviated as “3-α-isomaltosyl α,α-trehalose”, hereinafter) and 3-α-glucosyl α,α-trehalose represented by the chemical formula 3 (abbreviated as “3-α-glucosyl α,α-trehalose”, hereinafter) are concrete examples included in the aforesaid 3-α-glycosyl α,α-trehaloses.

Although 3-α-isomaltosyl α,α-trehalose of the present invention can be chemically synthesized, it is preferably synthesized by an enzymatic reaction of transferring isomaltose to α,α-trehalose by α-1,3 transglycosylation. Regarding the method for producing 3-α-isomaltosyl α,α-trehalose by an enzymatic reaction, the method of allowing α-isomaltosyl-transferring enzyme, originated from microorganisms such as Bacillus globisporus C9 (FERM BP-7143), Bacillus globisporus C11 (FERM BP-7144), Bacillus globisporus N75 (FERM BP-7591), and Arthrobacter globiformis A19 (FERM BP-7590), which are disclosed in International Patent Publication WO 02/055708 A1 applied for by the same applicant as the present invention, to act on a solution containing α,α-trehalose and a saccharide having a glucose polymerization degree of 3 or higher and bearing both the α-1,6 glucosidic linkage as a linkage at the non-reducing end and the α-1,4 glucosidic linkage other than the linkage at the non-reducing end to specifically hydrolyze the linkage between α-isomaltosyl moiety and other glucosaccharide moiety and to transfer the α-isomaltosyl moiety to α,α-trehalose by α-1,3 transglycosylation, can be advantageously used.

In the present invention, commercially available α,α-trehaloses are preferably used. As a commercially available α,α-trehalose, “TREHA®”, a high purity hydrous crystalline α,α-trehalose product with a α,α-trehalose content of 98% or higher, commercialized by Hayashibara Shoji Inc., Okayama, Japan, can be advantageously used. Optionally, α,α-trehalose prepared by conventional methods, for example, by extracting from yeasts, collecting from culture of bacteria having α,α-trehalose-producing abilities, and allowing enzymes to act on starchy substances to form α,α-trehalose, can be advantageously used. A commercially available reagent grade panose, commercialized by Hayashibara Biochemical Laboratories Inc., Okayama, Japan, can be used as an α-isomaltosylglucosaccharide in the present invention. Optionally, panose can be prepared by conventional methods, for example, by hydrolyzing pullulan, a natural polysaccharide, by panose-forming α-amylase originated from a microorganism such as Thermoactinomyces vulgaris. Further, α-isomaltosylglucosaccharides such as panose, 4-α-isomaltosyl maltose, 4-α-isomaltosyl maltotriose, and the like can be prepared by allowing α-glucosidase capable of converting α-1,4 glucosidic linkage into α-1,6 glucosidic linkage and originated from microorganisms such as Aspergillus niger, Aspergillus saitoi, Mucor javanicus, Penicillium crysogenum, Candida tropicalis, etc. Furthermore, α-isomaltosylglucosaccharides prepared by α-isomaltosylglucosaccharide-forming enzyme originated from microorganisms such as Bacillus globisporus C9 (FERM BP-7143), Bacillus globisporus C11 (FERM BP-7144), Bacillus globisporus N75 (FERM BP-7591), and Arthrobacter globiformis A19 (FERM BP-7590), which are disclosed in International Patent Publication No. WO 02/055708 A1 applied for by the same applicant as the present invention, can be advantageously used.

Conditions for enzymatic reaction to produce 3-α-isomaltosyl α,α-trehalose can be arbitrarily selected as far as 3-α-isomaltosyl α,α-trehalose can be produced. To produce 3-α-isomaltosyl α,α-trehalose, it is preferable to add α-isomaltosyl-transferring enzyme in an amount of, usually, 0.1 unit or higher, desirably, 1 to 100 units/g-α-isomaltosylglucosaccharide to a solution containing α,α-trehalose and α-isomaltosylglucosaccharide under the conditions selected from a temperature of 20 to 80° C., pH of 3 to 9, and reaction time of 0.1 to 100 hours, desirably, about 1 to 70 hours. One unit of α-isomaltosyl-transferring enzyme used in the present invention is defined as the amount of enzyme which forms one μmol of glucose per one minute using 1% (w/v) panose solution as substrate under the conditions of 30° C. and pH6.0. In the resulting reaction mixture, 3-α-isomaltosyl α,α-trehalose and oligosaccharides where one or more α-isomaltosyl residue are bound via the α-1,3glucosidic linkages are formed. Usually, the reaction mixture also includes reducing saccharides such as glucose and maltooligosaccharides, a cyclic tetrasaccharide having a structure of cyclof {→6)-α-D-glucopyranosyl-(1→3)-α-D-glucopyranosyl-(1→6)-α-D-glucopyranosyl-(1→3)-α-D-glucopyranosyl-(1→} (hereinafter, abbreviated as “cyclic tetrasaccharide” in this specification), and remaining intact α,α-trehalose, etc.

Although 3-α-glucosyl α,α-trehalose can be chemically synthesized, the saccharide ban be easily produced by allowing glucoamylase (EC3.2.1.3)to act on a solution containing 3-α-isomaltosyl α,α-trehalose, which is obtained above, to specifically hydrolyze the α-1,6 glucosidic linkage of 3-α-isomaltosyl α,α-trehalose.

In the present invention, glucoamylase usable for preparing 3-α-glucosyl α,α-trehalose from 3-α-isomaltosyl α,α-trehalose is not restricted as far as the enzyme easily hydrolyzes α-1,6 glucosidic linkage and hardly hydrolyzes α-1,3 glucosidic linkage. For example, glucoamylase originated from microorganisms such as Aspergillus niger and Rhizopus niveus can be advantageously used.

Conditions for enzymatic reaction to act glucoamylase can be arbitrarily selected as far as 3-α-glucosyl α,α-trehalose can be produced. To produce 3-α-gluosyl α,α-trehalose, it is preferable to add glucoamylase in an amount of, usually, 10 units or higher, desirably, 100 to 5,000 units per gram saccharide to a solution containing 3-α-isomaltosyl α,α-trehalose under the conditions selected from a temperature of 20 to 80° C., pH of 3 to 9, and reaction time of 0.1 to 100 hours, desirably, about 1 to 70 hours. One unit of glucoamylase activity used in the present invention is defined as the amount of enzyme which forms 10 mg of glucose per 30 minutes using 1% (w/v) soluble starch as a substrate under the conditions of 40° C. and pH4.5. By the enzymatic reaction, 3-α-glucosyl α,α-trehalose and oligosaccharides where one or more α-nigerosyl residue are bound via the α-1,6 glucosidic linkages are formed. Usually, the reaction mixture also includes reducing saccharides such as glucose and nigerose, cyclic tetrasaccharide, remaining intact α,α-trehalose, etc.

Various saccharide-derivatives having a structure of binding 3-α-isomaltosyl α,α-trehalose and/or 3-α-glucosyl α,α-trhalose and other saccharides, i.e. aforesaid 3-α-glycosyl α,α-trehaloses can be easily synthesized enzymatically by adding a proper saccharide and an enzyme catalyzing transglycosylation, for example, starchy substance and α-amylase, starchy substance and cyclodextrin glucanotransferase, lactose and β-galactosidase, etc., to a solution comprising 3-α-isomaltosyl α,α-trehalose and/or 3-α-glucosyl α,α-trehalose as an acceptor. In the case of using starchy substance and α-amylase, or starchy substance and cyclodextrin glucanotransferase for the reaction, 3-α-glycosyl α,α-trehaloses having a structure of binding maltooligosaccharide and an isomaltosyl residue of 3-α-isomaltosyl α,α-trehalose, a glucosyl residue of 3-α-glucosyl α,α-trehalose or a glucosyl residue constructing α,α-trehalose structure of those via the α-glucosidic linkage can be enzymatically synthesized. Also, in the case of using lactose and -galactosidase for the reaction, heterogeneous 3-α-glycosyl α,α-trehaloses composed of glucose and galactose, having a structure of binding galactose and an isomaltosyl residue of 3-α-isomaltosyl α,α-trehalose or a glucosyl residue of 3-α-glucosyl α,α-trehalose via β-galactosidic linkage, can be synthesized.

A solution comprising 3-α-glycosyl α,α-trehaloses, prepared by the above mentioned enzyme reaction, usually contains 5-15 w/w % (hereinafter, “w/w %” is abbreviated as “%” in this specification unless specified otherwise) of 3-α-glycosyl α,α-trehaloses, on a dry solid basis. 3-α-Glycosyl α,α-trehaloses can be arbitrarily used in the form of liquid or syrup after filtrating and purifying the above solution and in the form of a solid after drying.

Optionally, the above solution can be purified to make into a high 3-α-glycosyl α,α-trehaloses content product for increasing the content of 3-α-glycosyl α,α-trehaloses. For the purification methods, methods for separating and eliminating contaminant saccharides, for example, fermentation by yeast, membrane filtration, separatory sedimentation, an alkaline-treatment, can be arbitrarily used. Particularly, a method for eliminating contaminant saccharides and collecting high 3-α-glycosyl α,α-trehaloses content fractions by a column chromatography using a strongly acidic cation exchange resin in a salt form, disclosed in Japanese Patent Kokoku No. 50,477/87, Japanese Patent Kokai No. 50,319/92, etc., can be advantageously used. For the chromatography, fixed-bed method, moving-bed method, and semi-moving-bed method can be arbitrarily used.

Optionally, a saccharide composition comprising 3-α-glycosyl α,α-trehaloses, which shows substantially no reducibility, can be advantageously produced by hydrogenating a saccharide composition comprising 3-α-glycosyl α,α-trehaloses to eliminate its reducibility by converting reducing saccharides such as glucose and maltose, comprised in the saccharide composition.

3-α-Glycosyl α,α-trehaloses of the present invention have no reducibility in their intact forms and are extremely stable. They have a low sweetness but high in quality and mildness, and a satisfactory chemical stability. They can be used for stabilizing amino acids and peptides, which easily case browning by reacting with saccharides, and biologically active substances, which easily lose effective components and activities. Further, they have various properties such as osmotic pressure-regulating property, filling property, gloss-imparting property, moisture-retaining property, viscosity, crystallization-preventing property of other saccharides, hardly fermentable property, starch retrogradation-preventing property, etc.

These various properties of 3-α-glycosyl α,α-trehaloses can be advantageously used for various compositions for oral use including foods and beverages, nonessential grocery foods, feeds, and baits, foods and beverages, cosmetics, pharmaceuticals, etc. Particularly, various compositions can be advantageously produced by incorporating 3-α-glycosyl α,α-trehaloses with one or more other ingredients selected from the group consisting of non-reducing oligosaccharides, reducing oligosaccharides, sugar alcohols, and minerals into objective compositions.

The saccharide compositions comprising 3-α-glycosyl α,α-trehalose and the high 3-α-glycosyl α,α-trehalose content products, obtainable by purifying the saccharide composition, of the present invention can be used intact as a seasoning for sweetening. Optionally, they can be used after admixing with a suitable amount of one or more other sweeteners selected from the group consisting of powdery starch hydrolyzate, glucose, maltose, α,α-trehalose, sucrose, lactosucrose, isomerized sugar, honey, maple sugar, sorbitol, maltitol, lactitol, dihydrochalcone, stevioside, α-glycosyl stevioside, rebaudioside, glycyrrhizinate, L-aspartyl-L-phenylalanine-methyl ester, saccharin, glycine, and alanine. Further, they can be used in a mixture form with filler such as dextrin, starch, and lactose.

Further, powdery products of the saccharide composition comprising 3-α-glycosyl α,α-trehaloses and the high 3-α-glycosyl α,α-trehaloses content products, obtainable by purifying the saccharide composition, of the present invention can be arbitrarily used intact or in a mixture form with fillers, excipients, binders, etc. by shaping into various shapes such as granules, spheres, rods, plates, cubes, and tablets.

Since the sweetness of the saccharide composition comprising 3-α-glycosyl α,α-trehaloses and the high 3-α-glycosyl α,α-trehaloses content products, obtainable by purifying the saccharide composition, of the present invention well harmonizes with other tastes such as sourness, salty taste, astringency, delicious taste, and bitterness and they have satisfactory acid resistance and thermal stability, they can be advantageously used for sweetening, taste-improving, and quality-improving general foods and beverages.

For example, saccharide compositions comprising 3-α-glycosyl α,α-trehalose and 3-α-glycosyl α,α-trehalose high content products which can be obtained from the same, can be advantageously used as various seasonings such as soy sauce, powdered soy sauce, “miso” (bean paste), “funmatsu-miso” (a powdered miso), “moromi” (a refined sake), “hishio” (a refined soy sauce), “furikake” (a seasoned fish meal), mayonnaise, dressing, vinegar, “sanbai-zu” (a sauce of sugar, soy sauce and vinegar), “funmatsu-sushi-zu” (powdered vinegar for sushi), “chuka-no-moto” (an instant mix for Chinese dish), “tentsuyu” (a sauce for Japanese deep fat fried food), “mentsuyu” (a sauce for Japanese vermicelli), sauce, ketchup, “takuan-zuke-no-moto” (a premix for pickled radish), and “hakusai-zuke-no-moto” (a premix for fresh white rape pickles), “yakiniku-no-tare” (a sauce for Japanese grilled meat), curry roux, instant stew mix, instant soup mix, “dashi-no-moto” (an instant stock mix), mixed seasoning, “mirin” (a sweet sake), “shin-mirin” (a synthetic mirin), table sugar, and coffee sugar; various“wagashi” (Japanese confectionaries) such as “senbei” (a rice cracker), “arare” (a rice cake cube), “okoshi” (a millet and rice cake),“mochi” (a rise paste) and the like, “manju” (a bun with a bean-jam), “uiro” (a sweet rice jelly), “an” (a bean-jam) and the like, “yokan” (a sweet jelly of beans), “mizu-yokan” (a soft azuki-bean jelly), “kingyoku” (a kind of yokan), jelly, pao de Castella, and “amedama” (a Japanese toffee); western confectioneries such as a bun, biscuit, cracker, cookie, pie, pudding, butter cream, custard cream, cream puff, waffle, sponge cake, doughnut, chocolate, chewing gum, caramel, and candy; frozen desserts such as an ice cream and sherbet; syrups such as a “kajitsu-no-syrup-zuke” (a preserved fruit) and “korimitsu” (a sugar syrup for shaved ice); pastes such as a flour paste, peanut paste, fruit paste, and spread; processed fruits and vegetables such as a jam, marmalade, “syrup-zuke” (fruit pickles), and “toka” (conserves); pickles and pickled products such as a “fukujin-zuke” (red colored radish pickles), “bettara-zuke” (a kind of whole fresh radish pickles), “senmai-zuke” (a kind of sliced fresh radish pickles), and “rakkyo-zuke” (pickled shallots); meat products such as a ham and sausage; products of fish meat such as a fish ham, fish sausage, “kamaboko” (a steamed fish paste), “chikuwa” (a kind of fish paste), and “tempura” (a Japanese deep-fat fried fish paste); “chinmi” (relish) such as a “uni” (urchin), “ika-no-shiokara” (salted guts of squid), “su-konbu” (processed tangle), “saki-surume” (dried squid strips), “fugu-no-mirin-boshi” (a dried mirin-seasoned swellfish); “tsukudani” (foods boiled down in soy sauce) such as those of laver, edible wild plants, dried squid, small fish, and shellfish; daily dishes such as a “nimame” (cooked beans), potato salad, and “konbu-maki” (a tangle roll); canned and bottled products such as those of milk product, fish meat, meat, fruit, and vegetable; alcoholic beverages such as a sake, synthetic sake, liqueur, and western liquor; soft drinks such as a coffee, tea, cocoa, juice, carbonated beverage, sour milk beverage, beverage containing a lactic acid bacterium; instant food products such as instant pudding mix, instant hot cake mix, “sokuseki-shiruko” (an instant mix of azuki-bean soup with rice cake), and instant soup mix; solid foods for babies; foods for therapy; and health drinks.

Also, they can be advantageously used for the purpose of improving preference of feeds or pet foods for domestic creatures, for example, domestic animals, poultry, honey bees, silkworms, and fishes.

Also, they can be advantageously used as a sweetener, taste-improving agent, taste-changing agent, and quality-improving agent for various compositions such as preferences, cosmetics, and pharmaceuticals in the form of a solid, paste, or liquid, for example, tobacco, cigarette, tooth paste, lipstick, rouge, lip cream, internal liquid medicine, tablet, troche, cod-liver oil in the form of drop, oral refrigerant, cachou, and gargle (mouthwash).

When used as a quality-improving agent or stabilizer, they can be advantageously used in biologically active substances susceptible to lose their effective components and activities, as well as in health foods and pharmaceuticals containing the biologically active substances. Examples of such biologically active substances are liquid preparations containing lymphokines such as α-, β-, and γ-interferons, tumor necrosis factor-α (TNF-α), tumor necrosis factor-β(TNF-β), macropharge migration inhibitory factor, colony-stimulating factor, transfer factor, and interleukin 2; liquid preparations containing hormone such as insulin, growth hormone, prolactin, erythropoietin, follicle-stimulating hormone, and placenta hormone; biological preparations such as BCG vaccine, Japanese encephalitis vaccine, measles vaccine, live polio vaccine, small pox vaccine, tetanus toxoid, Trimeresurus antitoxin, and human immunoglobulin; liquid preparations containing antibiotics such as penicillin, erythromycin, chloramphenicol, tetracycline, streptmycin, and kanamycin sulfate; liquid preparations containing vitamins such as thiamin, riboflavin, L-ascorbic acid, cod liver oil, carotenoide, ergosterol, tocopherol; solutions of enzymes such as lipase, elastase, urokinase, protease, β-amylase, isoamylase, glucanase, and lactase; extracts such as ginseng extract, turtle extract, chlorella extract, aloe extract, and propolis extract; living microorganisms such as virus, lactic acid bacteria, and yeast; and royal jelly. These biologically active substances can be easily prepared into health foods and pharmaceuticals, which have a satisfactorily-high stability and quality with less fear of losing or inactivating their effective components and activities by using them.

The methods for incorporating a saccharide composition comprising 3-α-glycosyl α,α-trehalose or a 3-α-glycosyl α,α-trehalose high content product, obtained from the same, into the aforesaid compositions are those which can incorporate them into a variety of compositions before completion of their processing, and which can be appropriately selected among the following conventional methods; mixing, dissolving, melting, soaking, penetrating, dispersing, applying, coating, spraying, injecting, crystallizing, and solidifying. The amount of them to be preferably incorporated into the final compositions is usually an amount of 0.1% or higher, desirably, 0.5% or higher.

The following experiments concretely explain the present invention.

### EXPERIMENT 1

Preparation of α-isomaltosyl-transferring Enzyme

Experiment 1-1

Cultivation of Bacillus globisporus C11

A liquid culture medium consisting of 4% (w/v) of “PINE-DEX #4, a partial starch hydrolyzate, 1.8% (w/v) of “ASAHIMEAST”, a yeast extract, 0.1% (w/v) of dipotassium phosphate, 0.06% (w/v) of sodium phosphate dodeca-hydrate, 0.05% (w/v) of magnesium sulfate hepta-hydrate, and water was placed in 500-ml Erlenmeyer flasks in an amount of 100 ml each, sterilized by autoclaving at 121° C. for 20 min, cooled and seeded with Bacillus globisporus C11 strain, followed by culturing under rotary-shaking conditions at 27° C. and 230 rpm for 48 hours for a seed culture. About 20 L of a fresh preparation of the same liquid culture medium as used in the above seed culture were placed in a 30 L-fermenter, sterilized by heating, and then cooled to 27° C., and inoculated with 1% (v/v) of the seed culture, followed by culturing at 27° C. and pH 6.0 to 8.0 for 48 hours under aeration-agitation conditions. After the completion of the culture, about 0.55 unit/ml of α-isomaltosylglucosaccharide-forming enzyme activity and about 1.8 units/ml of α-isomaltosyl-transferring enzyme activity were detected in the resulting culture by measuring their enzyme activities. About 18 L of a supernatant obtained by centrifugation at 10,000 rpm for 30 min had 0.51 unit/ml of α-isomaltosylglucosaccharide-forming enzyme activity, i.e., a total enzymatic activity of about 9,180 units; and about 1.7 units/ml of α-isomaltosyl-transferring enzyme activity, i.e., a total enzymatic activity of about 30,400 units.

The enzymatic activities of the above two enzymes were assayed as follows: The activity of α-isomaltosylglucosaccharide-forming enzyme was measured by the following assay: A substrate solution was prepared by dissolving maltotriose in 100 mM acetate buffer (pH 6.0) to give a concentration of 2% (w/v). A reaction mixture was prepared by mixing 0.5 ml of the substrate solution and 0.5 ml of an enzyme solution, and incubated at 35° C. for 60 min. After stopping the reaction by boiling for 10 min, the amount of glucose formed in the reaction mixture was determined by high-performance liquid chromatography (HPLC). One unit of α-isomaltosylglucosaccharide-forming activity was defined as the amount of the enzyme that forms one μmole of maltose per minute under the above conditions. HPLC was carried out using “SHODEX KS-801 column”, Showa Denko K.K., Tokyo, Japan, at a column temperature of 60° C. and a flow rate of 0.5 ml/min of water, and using “RI-8012”, a differential refractometer commercialized by Tosho Corporation, Tokyo, Japan.

The activity of α-isomaltosyl-transferring enzyme was measured by the following assay: A substrate solution was prepared by dissolving panose in 100 mM acetate buffer (pH 6.0) to give a concentration of 2% (w/v). A reaction mixture was prepared by mixing 0.5 ml of the substrate solution and 0.5 ml of an enzyme solution, and incubated at 60° C. for 30 min. After stopping the reaction by boiling for 10 min, the amount of glucose formed in the reaction mixture was determined by the glucose oxidase-peroxidase method. One unit of α-isomaltosyl-transferring activity was defined as the amount of the enzyme that forms one μmole of glucose per minute under the above conditions.

Experiment 1-2

Preparation of a Partially Purified Enzyme

About 18 L of the culture supernatant, obtained by the method of Experiment 1-1, were salted out with 80% saturated ammonium sulfate solution and allowed to stand at 4° C. for 24 hours, and the formed precipitates were collected by centrifugation at 10,000 rpm for 30 min, dissolved in 10 mM sodium phosphate buffer (pH 7.5), and dialyzed against the same buffer to obtain about 416 ml of a crude enzyme solution. The crude enzyme solution had about 8,440 units of α-isomaltosylglucosaccharide-forming enzyme and about 28,000 units of α-isomaltosyl-transferring enzyme. The crude enzyme solution was subjected to ion-exchange column chromatography using “SEPABEADS FP-DA13” gel, an ion-exchanger commercialized by Mitsubishi Chemical Corporation, Tokyo, Japan. Bothα-isomaltosylglucosaccharide-forming enzyme and α-isomaltosyl-transferring enzyme were eluted as non-adsorbed fractions without adsorbing on the “SEPABEADS FP-DA13” gel. The non-adsorbed fraction was collected and dialyzed against 10 mM sodium phosphate buffer (pH 7.0) with 1 M ammonium sulfate. The dialyzed solution was centrifuged to remove impurities, and subjected to affinity column chromatography using 500 ml of “SEPHACRYL HR S-200” gel, a gel commercialized by Amersham Corp. Div. Amersham International, Arlington heights, Ill., USA. Enzymatically active components were adsorbed on “SEPHACRY HR S-200” gel and, when sequentially eluted with a linear gradient decreasing from 1 M to 0 M of ammonium sulfate and a linear gradient increasing from 0 mM to 100 mM of maltotetraose, the α-isomaltosyl-transferring enzyme and the α-isomaltosylglucosaccharide-forming enzyme were separately eluted, i.e., the former was eluted with a linear gradient of ammonium sulfate at about 0.3 M and the latter was eluted with a linear gradient of maltotetraose at about 30 mM. Thus, fractions with the α-isomaltosylglucosaccharide-forming enzyme activity and those with the α-isomaltosyl-transferring enzyme activity were separately collected as partially purified enzyme preparations of α-isomaltosylglucosaccharide-forming enzyme and α-isomaltosyl-transferring enzyme. Further, the enzymes were respectively purified.

Experiment 1-3

Purification of α-isomaltosylglucosaccharide-forming Enzyme

The partially purified enzyme preparation having α-isomaltosylglucosaccharide-forming enzyme activity, obtained by the method in Experiment 1-2, was dialyzed against 10 mM sodium phosphate buffer (pH 7.0) with 1 M ammonium sulfate. The dialyzed solution was centrifuged to remove impurities, and subjected to hydrophobic column chromatography using 350 ml of “BUTYL-TOYOPEARL 650M” gel, a hydrophobic gel commercialized by Tosho Corporation, Tokyo, Japan. The enzyme was adsorbed on the “BUTYL-TOYOPEARL 650M” gel and, when eluted with a linear gradient decreasing from 1 M to OM of ammonium sulfate, the enzymatic activity was eluted with a linear gradient of ammonium sulfate at about 0.3 M, and fractions with the enzyme activity were collected. The collected solution was dialyzed against 10 mM sodium phosphate buffer (pH 7.0) with 1 M ammonium sulfate, and the dialyzed solution was centrifuged to remove impurities, and purified by affinity chromatography using “SEPHACRYL HR S-200” gel. The amount of enzyme activity, specific activity and yield of the α-isomaltosylglucosaccharide-forming enzyme in each purification step are in Table 1.

TABLE 1

| | IMG* | Specific activity | |
|---|---|---|---|
| | activity | of IMG* | Yield |
| Purification step | (units) | (units/mg-protein) | (%) |
| Culture supernatant | 9,180 | 0.1 | 100 |
| Dialyzed solution after | 8,440 | 0.6 | 91.9 |
| salting out with | | | |
| ammonium sulfate | | | |
| Eluate from ion-exchange | 6,620 | 1.1 | 72.1 |
| column chromatography | | | |
| Eluate from 1st affinity | 4,130 | 8.8 | 45.0 |
| column chromatography | | | |
| Eluate from hydrophobic | 3,310 | 11.0 | 36.1 |
| column chromatography | | | |
| Eluate from 2nd affinity | 2,000 | 13.4 | 21.8 |
| column chromatography | | | |

*“IMG” means α-isomaltosylglucosaccharide-forming enzyme.

The finally purified α-isomaltosylglucosaccharide-forming enzyme specimen was assayed for purity on gel electrophoresis using a 7.5% (w/v) polyacrylamide gel and detected on the gel as a single protein band, i.e., a high purity specimen.

Experiment 1-4

Purification of α-isomaltosyl-transferring Enzyme

The partially purified enzyme preparation having an α-isomaltosyl-transferring enzyme activity, obtained by the method in Experiment 1-2, was dialyzed against 10 mM sodium phosphate buffer (pH 7.0) with 1 M ammonium sulfate. The dialyzed solution was centrifuged to remove impurities, and subjected to hydrophobic column chromatography using 350 ml of “BUTYL-TOYOPEARL 650M” gel, a hydrophobic gel commercialized by Tosho Corporation, Tokyo, Japan. The enzyme adsorbed on the “BUTYL-TOYOPEARL 650M” gel and, when eluted with a linear gradient decreasing from 1 M to 0 M of ammonium sulfate, the enzymatically active fractions were eluted with a linear gradient of ammonium sulfate at about 0.3 M, and fractions with the enzyme activity were collected. The collected solution was dialyzed against 10 mM sodium phosphate buffer (pH 7.0) with 1 M ammonium sulfate, and the dialyzed solution was centrifuged to remove impurities, and purified by affinity chromatography using “SEPHACRYL HR S-200” gel. The amount of enzyme activity, specific activity and yield of the α-isomaltosyl-transferring enzyme in each purification step are in Table 2.

TABLE 2

| | IMT* | Specific activity | |
|---|---|---|---|
| | activity | of IMT* | Yield |
| Purification step | (units) | (units/mg-protein) | (%) |
| Culture supernatant | 30,400 | 0.45 | 100 |
| Dialyzed solution after | 28,000 | 1.98 | 92.1 |
| salting out with ammonium | | | |
| sulfate | | | |
| Eluate from ion-exchange | 21,800 | 3.56 | 71.7 |
| column chromatography | | | |
| Eluate from 1st affinity | 13,700 | 21.9 | 45.1 |
| column chromatography | | | |
| Eluate from hydrophobic | 10,300 | 23.4 | 33.9 |
| column chromatography | | | |
| Eluate from 2nd affinity | 5,510 | 29.6 | 18.1 |
| column chromatography | | | |

*“IMT” means α-isomaltosyl-transferring enzyme.

The finally purified α-isomaltosyl-transferring enzyme specimen was assayed for purity on gel electrophoresis using a 7.5% (w/v) polyacrylamide gel and detected on the gel as a single protein band, i.e., a high purity specimen.

### EXPERIMENT 2

Formation of Transferred Saccharide A

One hundred milliliters of an aqueous solution containing, on a dry solid basis, 17.1 g of α,α-trehalose, 6.3 g of panose, and 50 mM acetate buffer (pH 6.0) was adjusted to 30° C. Then, five units/g-panose of the purified α-isomaltosyl-transferring enzyme, prepared by the method in Experiment 1-4, was admixed with the solution and subjected to enzymatic reaction at 30° C. for 24 hours. After the completion of the reaction, the reaction was stopped by heating at 100° C. for 10 minutes to inactivate the enzyme. The sugar composition of the resulting reaction mixture was analyzed by HPLC. The result of HPLC is in Table 3.

TABLE 3

| Elution time | | Sugar composition |
|---|---|---|
| on HPLC (min) | Saccharide | (%) |
| 44.2 | Transferred saccharide A | 13.7 |
| 51.5 | α,α-Trehalose | 66.6 |
| 58.9 | Glucose | 7.9 |
| 62.0 | Cyclic tetrasaccharide | 6.3 |
| — | Others | 5.5 |

As shown in Table 3, a transferred saccharide A (Rt: 44.2 min), which was considered to have been formed from α,α-trehalose and panose by the action of α-isomaltosyl-transferring enzyme (abbreviated as “Transferred saccharide A”, hereinafter), was detected along with the remaining intact α,α-trehalose (Rt: 51.5 min), as well as glucose (Rt: 58.9 min) and cyclic tetrasaccharide (Rt: 62.0 min) which were formed from panose by the action of α-isomaltosyl-transferring enzyme. The content of Transferred saccharide A was 13.7%. Taking account of the reaction specificity of α-isomaltosyl-transferring enzyme, the produced Transferred saccharide A was presumed to be 3-α-isomaltosyl α,α-trehalose where an α-isomaltosyl residue was bound with α,α-trehalose via the α-1,3 linkage.

HPLC was carried out using “MCI GEL CK04SS column”, Mitsubishi Chemical Corporation, Tokyo, Japan, two columns are connected tandem, at a column temperature of 80° C. and a flow rate of 0.4 ml/min of water, and using “RI-8012”, a differential refractometer commercialized by Tosho Corporation, Tokyo, Japan.

### EXPERIMENT 3

Purification of Transferred Saccharide A

Sodium hydroxide was admixed with about 100 ml of the reaction mixture, obtained by the method in Experiment 2, to adjust the pH to 12, and the mixture was kept at 98° C. for two hours to decompose reducing sugars such as glucose. Subsequently, the resulting reaction mixture was decolored and desalted using “DIAION SK-1B” and “DIAION WA30”, both of which were ion-exchange resins commercialized by Mitsubishi Chemical Corporation, Tokyo, Japan, and further desalted using “DIAION SK-1B” and “IRA411”, an anion exchange resin commercialized by Organo Corporation, Tokyo, Japan. The desalted solution was filtrated by conventional method and concentrated. The saccharide concentrate was measured, revealing that it contained 18.8 g of a dry solid. From the HPLC analysis of the concentrate, it was revealed that the it contained 15.7% of Transferred saccharide A, 76.6% of α,α-trehalose, 7.2% of cyclic tetrasaccharide, and 0.5% of other saccharides. Subsequently, the concentrate was subjected to preparative HPLC. α,α-Trehalose was eluted from the column at a retention time (Rt) of about 36 to 43 min, and Transferred saccharide A and cyclic tetrasaccharide were eluted at a Rt of 53 to 60 min. A fraction containing Transferred saccharide A and cyclic tetrasaccharide was collected and 3.6 g, d.s.b., of a partially purified Transferred saccharide A preparation was obtained. Upon HPLC analysis, the partially purified preparation contained 60.2% of Transferred saccharide A and 39.8% of cyclic tetrasaccharide. Preparative HPLC was carried out using “ODS-AQ R355-15AQ column”, YMC Co., Ltd., Kyoto, Japan, at a column temperature of 25° C. and a flow rate of 20 ml/min of water, and using “ERC-7530”, a differential refractometer commercialized by Erma Inc., Tokyo, Japan.

Successively, isomaltodextranase treatment was carried out to hydrolyze cyclic tetrasaccharide in the partially purified preparation into isomaltose. An aqueous solution containing 1% of the partially purified Transferred saccharide preparation was adjusted to pH 5.0 and a temperature of 50° C. and admixed with isomaltodextranase originated from Arthrobacter globiformis in an enzyme amount of 1,000 units/g-solid, and then enzymatically reacted at 50° C. for 24 hours. The reaction was stopped by heating at 100° C. for 10 min to inactivate the enzyme. The resulting reaction mixture was desalted and concentrated, and 3.4 g of hydrolyzates were obtained. Upon HPLC analysis, the hydrolyzate contained 56.7% of Transferred saccharide A and 43.3% of isomaltose. Isomaltodextranase originated from Arthrobacter globiformis was prepared according to the method described in Agricultural Biological Chemistry, Vol.52, pp.495-501 (1988).

Transferred saccharide A and isomaltose in the hydrolyzate were separated using the above preparative HPLC, and a fraction containing Transferred saccharide A was collected. The obtained fraction was filtrated and concentrated, and 1.6 g of purified Transferred saccharide A preparation was obtained. Upon HPLC analysis, it was revealed that the preparation contained 99.9% or higher of Transferred saccharide A. It was revealed that the preparation is a high purity Transferred saccharide A.

### EXPERIMENT 4

Structural Analyses of Transferred Saccharide A

Experiment 4-1

Mass Spectrometry

The mass of the purified Transferred saccharide A, obtained by the method in Experiment 3, was analyzed with an electro-spray-ionization method using “LCQ Advantage”, a mass spectrometer commercialized by Theremo Electron K.K., Kanagawa, Japan. A sodium-added molecular ion with a mass of 689 was remarkably detected and the data, revealing that the mass of the saccharide was 666.

Experiment 4-2

Component sugar

The component sugar was examined by hydrolyzing the purified Transferred saccharide A, obtained by the method in Experiment 3, with a diluted sulfuric acid according to conventional method and analyzed for the resulting hydrolyzate with gas chromatography. Only D-glucose was detected in the hydrolyzate and it was revealed that the saccharide was constructed with D-glucose.

Experiment 4-3

Methylation Analysis

After methylating the purified Transferred saccharide A, obtained by the method in Experiment 3, according to conventional method, the resulting methylated product was hydrolyzed with an acid, and the resulting hydrolyzates were reduced and acetylated. The resulting partially methylated hexitol acetates were analyzed by gas chromatography. As a result, 2,3,4,6-tetramethyl-1,5-diacetyl-glucitol, 2,4,6-trimethyl-1,3,5-tetraacetyl-glucitol, and 2,3,4-trimethyl-1,5,6-tetraacetly-glucitol were detected in a ratio of 1.9:1.1:1.0. It was revealed that the saccharide was constructed with two molecules of glucose residues whose hydroxyl groups at C1 position were involved in binding, one molecule of glucose reside whose hydroxyl groups at C1 and C3 positions were involved in binding, and one molecule of glucose residue whose hydroxyl groups at C1 and C6 positions were involved in binding.

Experiment 4-4

Nuclear Magnetic Resonance (NMR)

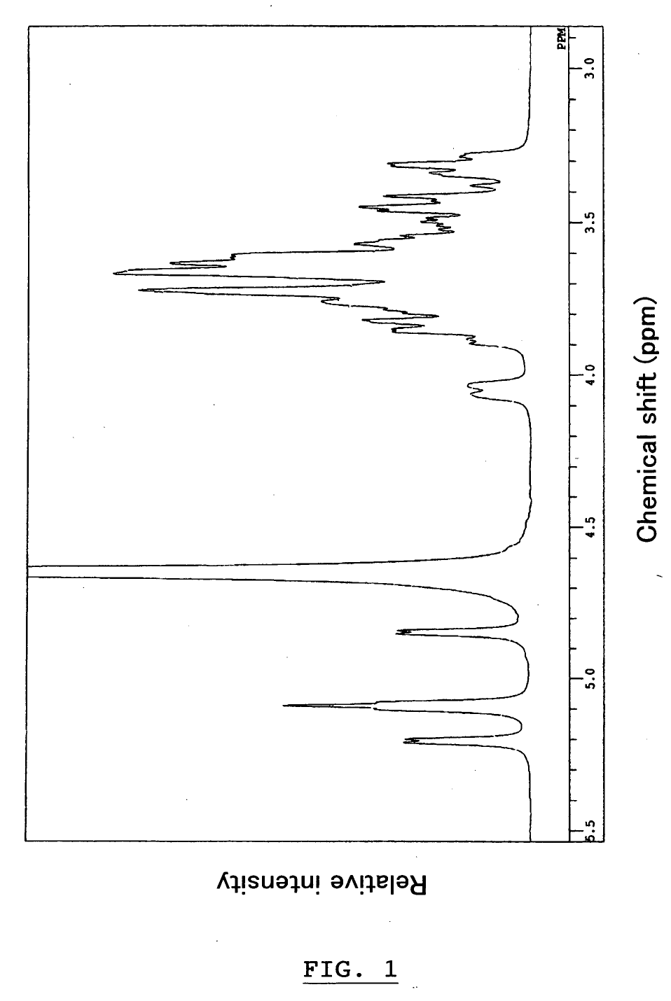

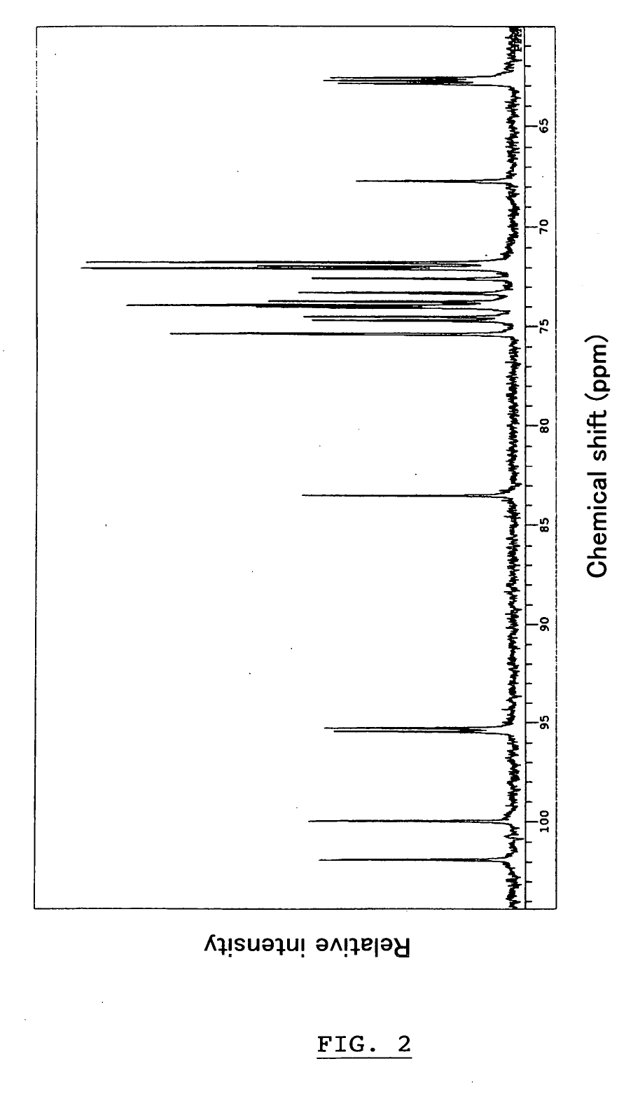

The purified transferred saccharide A was subjected to NMR analysis using “JMN-AL300”, a NMR apparatus commercialized by JOEL Inc., Mass., USA, and revealed its ¹H-NMR spectrum and ¹³C-NMR spectrum, shown in FIGS. 1 and 2, respectively. From the data of the spectra, chemical shifts of individual carbon atoms of the saccharide were assigned. The results are in Table 4.

TABLE 4

| Glucose residue | Carbon atom No. | Chemical shift (ppm) |
|---|---|---|
| a | 1 | 95.3 |
| | 2 | 73.3 |
| | 3 | 74.7 |
| | 4 | 72.0 |
| | 5 | 74.0 |
| | 6 | 62.9 |
| b | 1 | 95.5 |
| | 2 | 72.1 |
| | 3 | 83.5 |
| | 4 | 71.8 |
| | 5 | 74.5 |
| | 6 | 62.8 |
| c | 1 | 101.9 |
| | 2 | 73.8 |
| | 3 | 75.4 |
| | 4 | 71.8 |
| | 5 | 72.6 |
| | 6 | 67.7 |
| d | 1 | 100.0 |
| | 2 | 74.0 |
| | 3 | 75.4 |
| | 4 | 72.1 |
| | 5 | 74.1 |
| | 6 | 62.6 |

From the above data obtained by the structural analyses, it was revealed that Transferred saccharide A is a saccharide having a structure shown in FIG. 3, i.e., 3-α-isomaltosyl α,α-trehalose represented by the chemical formula 2.
O-α-D-Glcp-(1→6)-O-α-D-Glcp-(1→3)-O-α-D-Glcp-(1→1)-α-D-Glcp Chemical formula 2

### EXPERIMENT 5

Preparation of Partial Hydrolyzate B

An aqueous solution with a saccharide concentration of 2% was prepared using a half aliquot of the purified 3-α-isomaltosyl α,α-trehalose (0.8 gram, d.s.b.), prepared by the method of Experiment 3, and adjusted to pH4.5. Then, 3,000units/g-solid of “GLCOZYME#12000”, a glucoamylase preparation commercialized by Nagase & Co., Ltd., Osaka, Japan, was admixed with the solution, and followed by the enzyme reaction at 50° C. for 24 hours. After the completion of the reaction, the reaction was stopped by heating at 100° C. for 10 minutes to inactivate the enzyme. After filtrating and desalting the resulting reaction mixture, the sugar composition of the resulting solution was analyzed by HPLC. The result of HPLC is in Table 5.

TABLE 5

| Elution time | | Sugar composition |
|---|---|---|
| on HPLC (min) | Saccharide | (%) |
| 44.2 | 3-α-Isomaltosyl α,α-trehalose | 2.5 |
| 47.0 | Partial hydrolyzate B | 71.7 |
| 58.9 | Glucose | 25.8 |

As shown in Table 5, a partial hydrolyzate B (Rt: 47.0 min), which was considered to have been formed from 3-α-isomaltosyl α,α-trehalose by the action of glucoamylase (abbreviated as “Partial hydrolyzate B”, hereinafter), was detected with the remaining intact 3-α-isomaltosyl α,α-trehalose (Rt: 44.2 min) and glucose (Rt: 58.9 min) which were formed from 3-α-isomaltosyl α,α-trehalose by the action of glucoamylase. The content of Partial hydrolyzate B in the hydrolyzate was 71.7%. Taking account of the reaction specificity of glucoamylase, the produced Partial hydrolyzate B was presumed to be 3-α-glucosyl α,α-trehalose where a glucosyl residue was bound with α,α-trehalose via α-1,3 linkage. Subsequently, the hydrolyzate was subjected to preparative HPLC. Glucose, Partial hydrolyzate B, and the remaining 3-α-isomaltosyl α,α-trehalose were eluted from the column at Rts of about 34 to 48min, 44 to 48min and 53 to 60min, respectively. A fraction containing Partial hydrolyzate B was collected and 0.49 gram, d.s.b., of Partial hydrolyzate B was obtained. Upon HPLC analysis, it was revealed that the preparation contained 99.9% or higher of Partial hydrolyzate B. It was revealed that the preparation is a high purity Partial hydrolyzate B.

### EXPRERIMENT 6

Structural analyses of Partial Hydrolyzate B

Experiment 6-1

Mass Spectrometry

The mass of the purified Partial hydrolyzate B, obtained by the method in Experiment 5, was analyzed by the method in Experiment 4-1. A sodium-added molecular ion with a mass of 527 was remarkably detected, revealing that the mass of the saccharide was 504.

Experiment 6-2

Component Sugar

The component sugar was examined by hydrolyzing the purified Partial hydrolyzate B, obtained by the method in Experiment 5, with a diluted sulfuric acid according to conventional method, and analyzed for the resulting hydrolyzate with gas chromatography. Only D-glucose was detected in the hydrolyzate, revealing that the saccharide was constructed with D-glucose.

Experiment 6-3

Methylation Analysis

After methylating the purified Partial hydrolyzate B, obtained by the method in Experiment 5, according to conventional method, the resulting methylated product was hydrolyzed with an acid, and the resulting hydrolyzates were reduced and acetylated. The resulting partially methylated hexitol acetates were analyzed by gas chromatography. As a result, 2,3,4,6-tetramethyl-1,5-diacetyl-glucitol and 2,4,6-trimethyl-1,3,5-tetraacetyl-glucitol were detected in a ratio of 2.1:1.0. It was revealed that the saccharide was constructed with two molecules of glucose residues whose hydroxyl groups at C1 position were involved in binding and one molecule of glucose reside whose hydroxyl groups at C1 and C3 positions were involved in binding.

Experiment 6-4

Nuclear Magnetic Resonance (NMR)

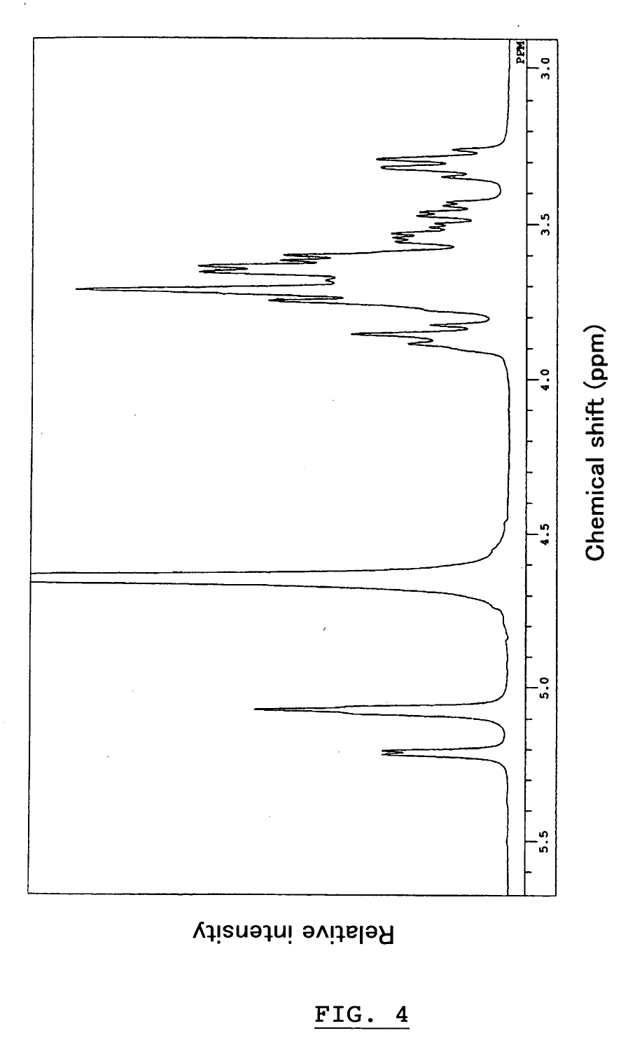

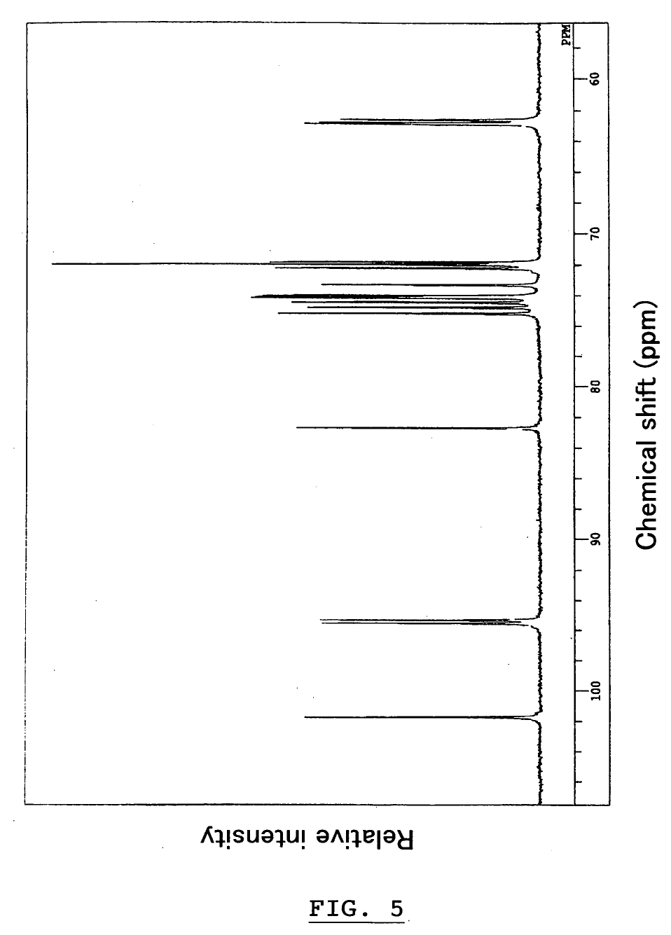

The purified Partial hydrolyzate B was subjected to NMR analysis and revealed its ¹H-NMR spectrum and ¹³C-NMR spectrum, shown in FIGS. 4 and 5, respectively. From the data of the spectra, chemical shifts of individual carbon atoms of the saccharide were assigned. The results are in Table 6.

TABLE 6

| Glucose residue | Carbon atom No. | Chemical shift (ppm) |
|---|---|---|
| a | 1 | 95.4 |
| | 2 | 73.3 |
| | 3 | 74.8 |
| | 4 | 72.0 |
| | 5 | 74.1 |
| | 6 | 62.9 |
| b | 1 | 95.6 |
| | 2 | 72.3 |
| | 3 | 82.7 |
| | 4 | 71.9 |
| | 5 | 74.5 |
| | 6 | 62.8 |
| c | 1 | 101.7 |
| | 2 | 74.1 |
| | 3 | 75.2 |
| | 4 | 72.0 |
| | 5 | 74.2 |
| | 6 | 62.6 |

From the above data obtained by the structural analyses, it was revealed that Partially hydrolyzate B is a saccharide having a structure shown in FIG. 6, i.e., 3-αglucosyl α,α-trehalose represented by the chemical formula 3.
O-α-D-Glcp-(1→6)-O-α-D-Glcp-(1→3)-O-α-D-Glcp Chemical formula 3

The following examples 1 to 8 explain 3-α-glycosyl α,α-trehaloses and the preparations thereof. Further, examples 9 to 19 explain compositions comprising 3-α-glycosyl α,α-trehaloses or saccharides comprising the same.

### EXAMPLE 1

“TREHA®”, α,α-trehalose commercialized by Hayashibara Shoji Inc., Okayama, Japan, and panose produced by Hayashibara Biochemical Laboratories Inc., Okayama, Japan, were added to water and dissolved to give respective concentrations of 25% and 9.2%, and then the solution was adjusted to pH 6.0 and 35° C. The solution was admixed with 2 units/g-panose of a partially purified enzyme preparation having α-isomaltosyl-transferring enzyme activity, prepared by the method in Experiment 1-2, and followed by the reaction for 48 hours. After heating and keeping the resulting reaction mixture to 95° C. for 10 minutes, the reaction mixture was cooled and filtrated. According to conventional methods, the resulting filtrate was purified by decoloring with activated charcoal and desalting with H- and OH-forms of ion exchange resins. Further, the resulting saccharide solution was concentrated, dried, and pulverized to obtain a powdery product comprising 3-α-isomaltosyl α,α-trehalose in a yield of about 91%, d.s.b.

The product contains, d.s.b., 8.0% of glucose, 66.3% of α,α-trehalose, 13.9% of 3-α-isomaltosyl α,α-trehalose, 6.0% of cyclic tetrasaccharide, and 5.8% of other saccharides. Since the product has a mild sweetness, adequate viscosity, moisture-retaining activity, and inclusion property, the product can be advantageously used for various compositions such as foods and beverages, cosmetics, and pharmaceuticals as a sweetener, taste-improving agent, quality-improving agent, syneresis-preventing agent, stabilizer, filler, including agent, base for powderization, or the like.

### EXAMPLE 2

After dissolving a powdery product comprising 3-α-isomaltosyl α,α-trehalose, obtained by the method in Example 1, and adjusting the concentration to 60%, the resulting saccharide solution was subjected to a column chromatography using “AMBERLITE CR-1310 (Na-form)”, a strong acidic cation-exchanger resin commercialized by Japan Organo Co., Ltd., Tokyo, Japan. The resin was packed into four-jacketed stainless steel columns having a diameter of 5.4 cm, which were then cascaded in series to give a total gel bed depth of 20 m. Under the conditions of keeping the inner column temperature at 60° C., the saccharide solution was fed to the columns in a volume of 5%(v/v) and fractionated by feeding to the columns hot water heated to 60° C. at an SV (space velocity) of 0.13 to obtain fractions comprising 3-α-isomaltosyl α,α-trehalose while monitoring the saccharide composition of eluate by HPLC. The collected fraction was desalted and concentrated, and a syrup comprising 3-α-isomaltosyl α,α-trehalose with a concentration of 70% was obtained in a yield of about 16%, d.s.b.

The product contains, d.s.b., 61.5% of 3-α-isomaltosyl α,α-trehalose, 38.3% of cyclic tetrasaccharide, and 0.2% of other saccharides. The product shows substantially no reducibility and hardly causes aminocarbonyl reaction. The product has a mild sweetness, adequate viscosity, moisture-retaining activity, and inclusion property, and can be advantageously used for various compositions such as foods and beverages, cosmetics, and pharmaceuticals as a sweetener, taste-improving agent, quality-improving agent, syneresis-preventing agent, stabilizer, filler, including agent, base for powderization, or the like.

### EXAMPLE 3

The syrup comprising 3-α-isomaltosyl α,α-trehalose, obtained by the method in Example 2, was diluted to give a concentration of 2% and adjusted to pH 5.0 and 50° C. The solution was admixed with 500 units/g-solid of isomaltodextranase, prepared by the method in Experiment 3, and followed by the reaction for 48 hours. After heating and keeping the resulting reaction mixture to 95° C. for 10 minutes, the reaction mixture was cooled and filtrated. According to conventional methods, the resulting filtrate was purified by decoloring with activated charcoal and desalting with H- and OH-forms of ion exchange resins. Further, the resulting saccharide solution was concentrated to give a concentration of 65% and subjected to a column chromatography using a strongly acidic cation exchange resin described in Example 2. 3-α-Isomaltosyl α,α-trehalose high content fraction was collected, desalted, dried, and pulverized to obtain a powdery 3-α-isomaltosyl α,α-trehalose high content product in a yield of about 48%, d.s.b.

The product contains, d.s.b., 98% of 3-α-isomaltosyl α,α-trehalose. The product shows substantially no reducibility and hardly causes amino carbonyl reaction. The product has a mild sweetness, adequate viscosity, moisture-retaining activity, and inclusion property, and can be advantageously used for various compositions such as foods and beverages, cosmetics, and pharmaceuticals as a sweetener, taste-improving agent, quality-improving agent, syneresis-preventing agent, stabilizer, filler, including agent, base for powderization, or the like.

### EXAMPLE 4

The powdery product comprising 3-α-isomaltosyl α,α-trehalose, obtained by the method in Example 1, was dissolved in hot water to give a concentration of 10% and adjusted to pH 4.5 and 50° C. The solution was admixed with 1,000 units/g-solid of “GLCOZYME”, a glucoamylase preparation commercialized by Nagase & Co., Ltd., Osaka, Japan, and followed by the reaction for 48 hours. After heating and keeping the resulting reaction mixture to 95° C. for 10 minutes, the reaction mixture was cooled and filtrated. According to conventional methods, the resulting filtrate was purified by decoloring with activated charcoal and desalting with H- and OH-forms of ion exchange resins. Further, the resulting saccharide solution was concentrated to give a concentration of 65% to obtain a syrup comprising 3-α-glucosyl α,α-trehalose in a yield of about 95%, d.s.b.

The product contains, d.s.b., 14.7% of glucose, 66.2% of α,α-trehalose, 11.0% of 3-α-glucosyl α,α-trehalose, 6.0% of cyclic tetrasaccharide, and 2.1% of other saccharides. Since the product has a mild sweetness, adequate viscosity, moisture-retaining activity, and inclusion property, the product can be advantageously used for various compositions such as foods and beverages, cosmetics, and pharmaceuticals as a sweetener, taste-improving agent, quality-improving agent, syneresis-preventing agent, stabilizer, filler, including agent, base for powderization, or the like.

### EXAMPLE 5

A syrup comprising 3-α-glucosyl α,α-trehalose, obtained by the method in Example 4, was used as a material and subjected to a column chromatography using a strongly acidic cation exchange resin described in Example 2. 3-α-Gluosyl α,α-trehalose high content fraction was collected, desalted, dried, and pulverized to obtain a powdery 3-α-glucosyl α,α-trehalose high content product in a yield of about 6%, d.s.b.

The product contains, d.s.b., 98% of 3-α-gluosyl α,α-trehalose. The product shows substantially no reducibility and hardly causes aminocarbonyl reaction. The product has a mild sweetness, adequate viscosity, moisture-retaining activity, and inclusion property, and can be advantageously used for various compositions such as foods and beverages, cosmetics, and pharmaceuticals as a sweetener, taste-improving agent, quality-improving agent, syneresis-preventing agent, stabilizer, filler, including agent, base for powderization, or the like.

### EXAMPLE 6

About 18 liters of culture supernatant, obtained by the method in Experiment 1-1, was concentrated using a UF-membrane and about one liter of the concentrated enzyme solution having 7.7 units/ml of α-isomaltosylglucosaccharide-forming enzyme and 25.3 units/ml of α-isomaltosyl-transferring enzyme was collected.

A starch suspension containing about 12.5% of “TREHA®”, α,α-trehalose commercialized by Hayashibara Shoji Inc., Okayama, Japan, and about 12.5% of tapioca starch was prepared and admixed with 0.2%/g-starch of “NEOSPITASE”, an α-amylase product commercialized by Nagase&Co., Ltd, Osaka, Japan, and then followed by the enzyme reaction at 85 to 90° C. for about 20 min. Subsequently, the starch solution was autoclaved at 120° C. for 20 min and rapidly cooled to about 35° C. and a liquefied starch solution comprising α,α-trehalose with a DE of about 2 was obtained. The liquefied starch solution was admixed with 0.3 ml/g-starch of the above concentrated enzyme solution and followed by the enzyme reaction at pH 6.0 and 35° C. for 48 hours. After keeping the resulting reaction mixture at 95° C. for 30 min, the reaction mixture was cooled and filtrated. According to conventional methods, the resulting filtrate was purified by decoloring with activated charcoal and desalting with H- and OH-forms of ion exchange resins. Further, the resulting saccharide solution was concentrated to give a concentration of 70% and then, a syrup comprising 3-α-isomaltosyl α,α-trehalose was obtained in a yield of about 90%, d.s.b.

The product contains, d.s.b., 2.7% of glucose, 40.2% of α,α-trehalose, 18.2% of 3-α-isomaltosyl α,α-trehalose, 9.8% of cyclic tetrasaccharide, and 29.1% of other saccharides. The product has a mild sweetness, adequate viscosity, moisture-retaining activity, and inclusion property and can be advantageously used for various compositions such as foods and beverages, cosmetics, and pharmaceuticals as a sweetener, taste-improving agent, quality-improving agent, syneresis-preventing agent, stabilizer, filler, including agent, base for powderization, or the like.

### EXAMPLE 7

The syrup comprising 3-α-isomaltosyl α,α-trehalose, obtained by the method in Example 6, was diluted to give a concentration of 30% and adjusted to pH 4.5 and 50° C. The solution was admixed with 1,000 units/g-solid of “GLCOZYME”, a glucoamylase preparation commercialized by Nagase & Co., Ltd., Osaka, Japan, and followed by the reaction for 48 hours. After heating and keeping the resulting reaction mixture to 95° C. for 10 minutes, the reaction mixture was cooled and filtrated. According to conventional methods, the resulting filtrate was purified by decoloring with activated charcoal and desalting with H- and OH-forms of ion exchange resins. Further, the resulting saccharide solution was concentrated and hydrogenated according to conventional method to convert reducing sugars into sugar alcohols. The resulting reaction mixture was decolored, desalted, concentrated, dried, and pulverized to obtain a powdery product comprising 3-α-glucosyl α,α-trehalose in a yield of about 85%, d.s.b.

The product contains, d.s.b., 18.3% of sorbitol, 42.1% of α,α-trehalose, 13.7% of 3-α-glucosyl α,α-trehalose, 9.9% of cyclic tetrasaccharide, and 16.0% of other sugar alcohols. The product shows substantially no reducibility and hardly causes aminocarbonyl reaction. The product has a mild sweetness, adequate viscosity, moisture-retaining activity, and inclusion property and can be advantageously used for various compositions such as foods and beverages, cosmetics, and pharmaceuticals as a sweetener, taste-improving agent, quality-improving agent, syneresis-preventing agent, stabilizer, filler, including agent, base for powderization, or the like.

### EXAMPLE 8

A saccharide solution containing about 25% of 3-α-glucosyl α,α-trehalose high content product, obtained by the method in Example 5, and about 25% of lactose was prepared and adjusted to pH 6.0 and 40° C. The solution was admixed with 3 units/g-lactose of “BIOLACTA”, a β-galactosidase preparation commercialized by Daiwa Kasei K.K., Shiga, Japan, and followed by the reaction at pH 6.0 and 40° C. for 48 hours. After heating and keeping the resulting reaction mixture to 95° C. for 10 minutes, the reaction mixture was cooled and filtrated. According to conventional methods, the resulting filtrate was purified by decoloring with activated charcoal and desalting with H- and OH-forms of ion exchange resins. Further, the resulting saccharide solution was concentrated to give a concentration of 70% to obtain a syrup comprising 4-β-galactosyl-3-α-glucosyl α,α-trehalose in a yield of about 90%, d.s.b.

The product contains, d.s.b., 22.7% of glucose, 3.4% of galactose, 5.9% of lactose, 35.1% of 3-α-glucosyl α,α-trehalose, 13.5% of 4-β-galactosyl-3-α-glucosyl α,α-trehalose, and 19.4% of other saccharides. The product has a mild sweetness, adequate viscosity, moisture-retaining activity, and inclusion property and can be advantageously used for various compositions such as foods and beverages, cosmetics, and pharmaceuticals as a sweetener, taste-improving agent, quality-improving agent, syneresis-preventing agent, stabilizer, filler, including agent, base for powderization, or the like.

### EXAMPLE 9

Sweetener

A granulated sweetener was prepared by mixing to homogeneity one part by weight of a powdery saccharide products, obtained by the method in Example 1, with 0.01 part by weight of “αG-SWEET®”, α-glycosyl-stevioside commercialized by Toyo Sugar Refining Co., Ltd., Tokyo, Japan, and 0.01 part by weight of “ASPARTAME®”, L-aspartyl-L-phenylalaninemethylester, and by applying a granulator. The product comprises 3-α-isomaltosyl α,α-trehalose and is a sweetener composition having a satisfactory sweetening taste and the sweetening power of about two-folds higher than sucrose. Also, the product is a stable sweetener without fear of deteriorating even when preserved under an ambient temperature.

### EXAMPLE 10

Hard Candy

Fifty parts by weight of a syrupy saccharide product comprising 3-α-isomaltosyl α,α-trehalose, obtained by the method in Example 6, was admixed with 100 parts by weight of a sucrose solution with a concentration of 55% and heated. The solution was concentrated by heating under reduced pressure to give a moisture content of less than 2%, admixed with 0.6 part by weight of citric acid and appropriate amounts of lemon-flavor and coloring, and shaped in a usual manner to make into hard candies. The products are stable and high-quality hard candies with a satisfactory crisp, color, taste, and flavor, no crystallization of sucrose, and low hygroscopicity.

### EXAMPLE 11

Chewing Gum

Three parts by weight of gum base was melted by heating to give a soft texture, admixed with two parts by weight of anhydrous crystalline maltitol, two parts by weight of xylitol, three parts by weight of a syrupy saccharide product comprising 3-α-glucosyl α,α-trehalose, obtained by the method in Example 4, and appropriate amounts of flavor and coloring, kneaded using a roll machine in a usual manner, shaped, and packed to obtain the chewing gum. The product has a satisfactory texture, taste, and flavor, and is suitable as a chewing gum with a low cariogenicity and low calorie.

### EXAMPLE 12

Powdery Peptide

Two parts by weight of a syrupy saccharide product comprising 3-α-isomaltosyl α,α-trehalose, obtained by the method in Example 2, was admixed with one part by weight of “HIGHNUT S”, a soybean peptide solution with a 40% concentration, commercialized by Fuji Oil Co., Ltd., Osaka, Japan, placed on a plastic container, dried under reduced pressure at 50° C., and pulverized to make into a powdery peptide product. The product has a satisfactory flavor and is useful as a premix and a material of low calorie confectionaries such as ice cream. Also, the product is useful as a hardly digestive dietary fiber, material for regulating intestinal function, and material for health foods.

### EXAMPLE 13

Bath Agent

One part by weight of orange peel juice and 10 parts by weight of a syrupy saccharide product comprising 3-α-isomaltosyl α,α-trehalose, obtained by the method in Example 2, were mixed, spray-dried, and pulverized to make into a powdery product comprising orange peel extract and 3-α-isomaltosyl α,α-trehalose. Five parts by weight of the powdery product, 90 parts by weight of “yakijio” (baked sodium chloride), two parts by weight of hydrous crystalline trehalose, one part by weight of silicic anhydride, and 0.5 part by weight of “αG-HESPERIDINE”, α-glucosyl hesperidine commercialized by Hayashibara Shoji, Inc., Okayama, Japan, were mixed to make into a bath agent. The product is a high-quality bath agent with a rich orange flavor, which provides a skin of velvety texture and prevents chill after taking a bath, and can be used to dilute 100 to 10,000-fold with warm water for taking a bath.

### EXAMPLE 14

Cosmetic Cream

Two parts by weight of polyoxyethylenglycol monostearate, five parts by weight of self-emulsified glycerin monostearate, two parts by weight of a syrupy saccharide product comprising 3-α-isomaltosyl α,α-trehalose in high content, obtained by the method in Example 2, one part by weight of “αG-RUTIN®”, α-glucosyl rutin commercialized by Hayashibara Co., Ltd., Okayama, Japan, one part by weight of fluid paraffin, 10 parts by weight of glycerin trioctanoate, and appropriate amount of preservative were dissolved by heating in a usual manner, admixed with two parts by weight of L-lactic acid, five parts by weight of 1,3-butylene glycol, and 66 parts by weight of purified water, and emulsified using a homogenizer. The mixture was further admixed with appropriate amount of perfume, and stirred to produce a cosmetic cream. The product has an anti-oxidation activity and high stability, and can be advantageously used as a high-quality sunburn preventive, skin-care agent, whitening agent, etc.

### EXAMPLE 15

Toothpaste

Forty-five parts by weight of calcium phosphate (CaHPO4), 1.5 parts by weight of sodium lauryl sulfate, 25 parts by weight of glycerin, 0.5 part by weight of polyoxyethylenesorbitan laurate, 13 parts by weight of a powdery product comprising 3-α-glucosyl α,α-trehalose, obtained by the method in Example 7, 0.02 part by weight of saccharin, 0.05 part by weight of a preservative, and 15 parts by weight of water were mixed to make into a toothpaste. The product is a toothpaste improved in unpleasant taste, having a satisfactory availability, without deteriorating the washing power of the detergent.

### EXAMPLE 16

Solid Preparation for Fluid Diet

A composition consisting of 200 parts by weight of a powdery saccharide product comprising 3-α-isomaltosyl α,α-trehalose, obtained by the method in Example 1, 100 parts by weight of hydrous crystalline trehalose, 200 parts by weight of a powdery product comprising maltotetraose in high content, 270 parts by weight of powderized egg yolk, 209 parts by weight of skim milk, 4.4 parts by weight of sodium chloride, 1.8 parts by weight of potassium chloride, four parts by weight of magnesium sulfate, 0.01 part by weight of thiamine, 0.1 part by weight of sodium L-ascorbate, 0.6 part by weight of vitamin E acetate, and 0.04 part by weight of nicotinamide was prepared, filled 25 grams each in dampproof laminate bags, and heat-sealed to obtain the captioned product.

The product is a solid preparation for fluid diet with a satisfactory stability. A bag of the product can be advantageously used for supplying energy to living body by dissolving into about 150 to 300 ml of water to produce a fluid diet and by using orally or by tube feeding to nose, stomach, or intestines.

### EXAMPLE 17

Tablet

Fifty parts by weight of aspirin, 14 parts by weight of a powdery saccharide product comprising 3-α-isomaltosyl α,α-trehalose in high content, obtained by the method in Example 3, and four parts by weight of corn starch were mixed to homogeneity and then solidified using a tablet machine to produce 680 mg/tablet of tablets with a thickness of 5.25 mm. The product, which prepared by using filling-ability of 3-α-isomaltosyl α,α-trehalose, shows no hygroscopicity and a satisfactory physical strength and collapsability in water.

### EXAMPLE 18

Sugar-coated Tablet

One hundred and fifty mg of a raw tablet, used as core, was sugar-coated to give about 230 mg of sugar-coated tablet by using solution 1 consisting of 40 parts by weight of a powdery saccharide product comprising 3-α-isomaltosyl α,α-trehalose, obtained by the method in Example 3, two parts by weight of pulullan (average molecular weight of 200,000), 30 parts by weight of water, 25 parts by weight of talc, and three parts by weight of titanic oxide. Subsequently, the resulting tablet was further sugar-coated by using solution 2 consisting of 65 parts by weight of a powdery crystalline cyclic tetrasaccharide, one part by weight of pullulan, and 34 parts by weight of water. Furthermore, the resulting sugar-coated tablet was polished using wax to produce a sugar coated tablet having a satisfactory gloss and appearance. The product has a superior shock tolerance and keeps the high quality for a long period of time.

### EXAMPLE 19

Salve for Curing Wound

Seventy parts by weight of a powdery saccharide product comprising 3-α-glucosyl α,α-trehalose in high content, prepared by the method in Example 5, 300 parts by weight of maltose, 30 parts by weight of distilled water, and 50 parts by weight of methanol comprising three parts by weight of iodine were mixed. Further, 200 parts by weight of an aqueous solution containing pullulan in an amount of 10% (w/v) was admixed with the above mixture to produce a salve for curing wounds, which has a adequate spread property and adherability.

The product is a high quality salve comprising 3-α-glucosyl α,α-trehalose with a high stability. In addition to the disinfectant activity of iodine, maltose comprised in the product can be used as an energy-supplementing agent for cells. Therefore, the use of the product enables to shorten the curing period and to cure wounds completely.

### INDUSTRIAL APPLICABILITY

The establishment of novel 3-α-glycosyl α,α-trehaloses, their preparation and use of the present invention has a great industrial significance in the fields of foods and beverages, cosmetics, and pharmaceuticals.

1. An 3-α-glycosyl α,α-trehalose which has an α-glucosyl α,α-trehalose structure, represented by the chemical formula 1, intermolecularly.
O-α-D-Glcp-(1→3)-O-α-D-Glcp-(1→1)-α-D-Glcp Chemical formula 1

2. The 3-α-glycosyl α,α-trehalose of claim 1, wherein said 3-α-glycosyl α,α-trehalose is 3-isomaltosyl α,α-trehalose represented by the chemical formula 2.
O-α-D-Glcp-(1→3)-O-α-D-Glcp-(1→1)-α-D-Glcp Chemical formula 2

3. The 3-α-glycosyl α,α-trehalose of claim 1, wherein said 3-α-glycosyl α,α-trehalose is 3-α-glucosyl α,α-trehalose represented by the chemical formula 3.
O-α-D-Glcp-(1→3)-O-α-D-Glcp-(1→1)-α-D-Glcp Chemical formula 3

4. A method for forming 3-α-glycosyl α,α-trehalose of claim 1, which comprises a step of allowing α-isomaltosyl-transferring enzyme to act on an aqueous solution comprising α,α-trehalose and a saccharide having a glucose polymerization degree of 3 or higher and bearing both the α-1,6 glucosidic linkage as a linkage at the non-reducing end and the α-1,4 glucosidic linkage other than the linkage at the non-reducing end.

5. The method of claim 4, wherein said saccharide is prepared by allowing α-isomaltosylglucosaccharide-forming enzyme to act on partial starch hydrolyzates.

6. The method of claim 4, which further comprises a step of allowing glucoamylase to act on the reaction mixture.

7. A method of forming α-glycosyl α,α-trehalose, which comprises the step of allowing a saccharide-transferring enzyme to act on an aqueous solution comprising 3-α-isomaltosyl α,α-trehalose represented by the chemical formula 2 and/or 3-α-glucosyl α,α-trehalose represented by the chemical formula 3 and optional other saccharides to form said α-glycosyl α,α-trehalose of claim 1.

8. A process for producing 3-α-glycosyl α,α-trehalose of claim 2, which comprises the steps of:
allowing α-isomaltosyl-transferring enzyme to act on an aqueous solution comprising α,α-trehalose and a saccharide having a glucose polymerization degree of 3 or higher and bearing both the α-1,6 glucosidic linkage as a linkage at the non-reducing end and the α-1,4 glucosidic linkage other than the linkage at the non-reducing end to form 3-α-isomaltosyl α,α-trehalose represented by the chemical formula 2; and
collecting the resulting 3-α-isomaltosyl α,α-trehalose.

9. The process of claim 8, wherein said saccharide is prepared by allowing α-isomaltosylglucosaccharide-forming enzyme to act on starchy substances.

10. A process for producing 3-α-glycosyl α,α-trehalose of claim 3, which comprises the steps of:
allowing α-isomaltosyl-transferring enzyme to act on an aqueous solution comprising α,α-trehalose and a saccharide having a glucose polymerization degree of 3 or higher and bearing both the α-1,6 glucosidic linkage as a linkage at the non-reducing end and the α-1,4 glucosidic linkage other than the linkage at the non-reducing end to form 3-α-isomaltosyl α,α-trehalose represented by the chemical formula 2;
successively allowing glucoamylase to act on the resulting 3-α-isomaltosyl α,α-trehalose to form 3-α-glucosyl α,α-trehalose represented by the chemical formula 3; and
collecting the resulting 3-α-glucosyl α,α-trehalose.

11. A process for producing α-glycosyl α,α-trehalose, which comprises the step of:
allowing a saccharide-transferring enzyme to act on an aqueous solution comprising 3-α-isomaltosyl α,α-trehalose represented by the chemical formula 2 and/or 3-α-glucosyl α,α-trehalose represented by the chemical formula 3 and optional other saccharides to form α-glycosyl α,α-trehalose of claim 1; and
collecting the resulting α-glycosyl α,α-trehalose.

12. (canceled)

13. A composition which comprises α-glycosyl α,α-trehalose of claim 1.

14. The composition of claim 13, where one or more ingredients selected from the group consisting of other non-reducing saccharides, reducing saccharides, sugar alcohols, and minerals are incorporated into said α-glycosyl α,α-trehalose.

15. The composition of claim 13, which is in the form of a product for oral use, food and beverage, cosmetic, or pharmaceutical.
